(19) United States
(12) Patent Application Publication
(10) Pub. No.: US 2016/0341649 A1
(43) Pub. Date: Nov. 24, 2016

(54) PARTICLE SCORE CALIBRATION

(71) Applicant: CAN TECHNOLOGIES, INC., Hopkins, MN (US)

(72) Inventor: Jayd Marshal Kittelson, Otsego, MN (US)

(73) Assignee: CAN TECHNOLOGIES, INC., Hopkins, MN (US)

(21) Appl. No.: 15/106,438

(22) PCT Filed: Dec. 19, 2014
(86) PCT No.: PCT/US14/71430

§ 371 Date: Jun. 20, 2016

Related U.S. Application Data
(60) Provisional application No. 61/919,258, filed on Dec. 20, 2013.

(51) Int. Cl. G01N 15/02; G01N 21/359; A01K 5/00; G01N 21/85; G01N 33/02; B01F 15/00; G01N 21/27; G01N 21/3563

(52) U.S. Cl. CPC G01N 15/0211; G01N 21/274; G01N 21/359; G01N 21/3563; G01N 21/85; G01N 33/02; B01F 15/00214; B01F 15/00272; A01K 5/001; G01N 2021/8592; G01N 2201/127; B01F 2215/0008

(57) ABSTRACT

A method for developing a calibration for a near infrared reflectance spectrophotometer to predict the particle score of an ingredient, the method comprising (a) sorting a plurality of plant matter samples by size by passing such samples through a screen and subsequently calculating a particle score for the samples based on the number of samples passing through the screen, (b) measuring the absorbance or reflectance of the plurality of plant matter samples using the spectrophotometer, and (c) correlating the particle score from step (a) with the measured absorbance or reflectance from step (b),

### RELATED APPLICATIONS

This application claims the benefit of U.S. Provisional Patent Application No. 61/919,258, entitled PARTICLE SCORE CALIBRATION, filed on Dec. 20, 2013, which is herein incorporated by reference in its entirety.

### TECHNICAL FIELD

The present disclosure relates generally to spectroscopy. Aspects of the disclosure are particularly directed to predicting a particle score of a forage sample using near infrared spectroscopy.

### BACKGROUND

It is known to determine the particle score of a forage sample using a particle scorer such as the Penn State Three-Sieve Forage Particle Separator model no. C24682N commercially available from Nasco Catalog Outlet Store of Fort Atkinson, Wis., USA. However, such known particle scorers may be somewhat imprecise. It is also known to determine the chemical properties of forage (e.g., percentage crude protein fat, ash, fiber, etc.) using a near infrared reflectance (NIR) spectrometer (spectrophotometer), such as the FOSS model no. NIRsys II 5000 near infrared reflectance spectrometer or the FOSS INFRAXACT near infrared reflectance spectrometer or the FOSS XDS NIR analyzer, or FOSS NIRS DS2500, all commercially available from FOSS of Eden Prairie, Minn., USA, also known as Metrohm NIRSystems of Metrohm AG, or the Bruker FT-NIR, commercially available from Bruker Corporation of Billerica, Mass., USA. However, such known NIR instruments may not be able to predict the particle score of forages with accuracy.

### BRIEF DESCRIPTION OF THE DRAWINGS

FIG. 1 is a perspective view of a three-sieve Penn State Particle Separator device according to an exemplary embodiment.

FIG. 2A is a perspective view of a two-sieve Penn State Particle Separator device according to an exemplary embodiment.

FIG. 2B is a perspective view of the two-sieve Penn State Particle Separator device of FIG. 2A.

FIG. 3 is a perspective view of an Alternative Particle Scorer device according to an exemplary embodiment.

FIG. 4 is a graph showing NIR predictive ability of the NIR calibration developed using an Alternative Particle Scorer device according to Example 1.

FIG. 5A is a graph showing NIR predictive ability of the NIR calibration developed using the top sieve of the Penn State Particle Separator device according to Example 1.

FIG. 5B is a graph showing NIR predictive ability of the NIR calibration developed using the middle sieve of the Penn State Particle Separator device according to Example 1.

FIG. 5C is a graph showing NIR predictive ability of the NIR calibration developed using the bottom sieve of the Penn State Particle Separator device according to Example 1.

FIG. 6 is a graph showing the verification of actual particle score determined using wet chemistry values from the Alternative Particle Scorer device versus the NIR predicted particle score developed using the Alternative Particle Scorer device according to Example 1.

FIG. 7A is a graph showing the verification of actual particle score determined using wet chemistry values from the Penn State Particle Separator device top sieve versus the NIR predicted particle score developed using the Penn State Particle Separator device top sieve according to Example 1.

FIG. 7B is a graph showing the verification of actual particle score determined using wet chemistry values from the Penn State Particle Separator device middle sieve versus the NIR predicted particle score developed using the Penn State Particle Separator device middle sieve according to Example 1.

FIG. 7C is a graph showing the verification of actual particle score determined using wet chemistry values from the Penn State Particle Separator device bottom sieve versus the NIR predicted particle score developed using the Penn State Particle Separator device bottom sieve according to Example 1.

FIG. 8 is a graph showing the correlation between actual particle scores and NIR predictions for the Alternative Particle Score method according to Example 1.

FIG. 9A is a graph showing the correlation between actual particle scores and NIR predictions for Penn State particle fraction measurement (bypass top sieve) according to Example 1.

FIG. 9B is a graph showing the correlation between actual particle scores and NIR predictions for Penn State particle fraction measurement (bypass middle sieve) according to Example 1.

FIG. 9C is a graph showing the correlation between actual particle scores and NIR predictions for Penn State particle fraction measurement (bypass bottom sieve) according to Example 1.

FIG. 10 is a flow diagram illustrating the processing of a create calibration component in accordance with some embodiments of the disclosed technology.

FIG. 11 is a flow diagram illustrating the processing of a construct database component in accordance with some embodiments of the disclosed technology.

FIG. 12 is a flow diagram illustrating the processing of a determine particle scores component in accordance with some embodiments of the disclosed technology.

FIG. 13 is a flow diagram illustrating some of the components that may be incorporated in at least some of the computer systems and other devices on which the system operates and interacts with in some examples.

### DETAILED DESCRIPTION

Systems and methods for calibrating a near infrared reflectance spectrophotometer are disclosed. In one aspect, a method for developing a calibration for a near infrared reflectance spectrophotometer is provided to predict the particle score of an ingredient, the method comprising: (a) sorting a plurality of plant matter samples by size by passing such samples through a screen and subsequently calculating a particle score for the samples based on the number of samples passing through the screen, (b) measuring the absorbance or reflectance of the plurality of plant matter samples using the spectrophotometer, and (c) correlating the particle score from step (a) with the measured absorbance or reflectance from step (b).

In another aspect, a near infrared reflectance calibration for predicting a particle score for a dry ingredient is provided, the calibration produced by a method comprising: (a) sorting a plurality of forage samples by chop length by passing such samples through a particle separator having at least one screen and subsequently calculating a particle score for the samples based on the weight of the samples passing through the screen, (b) measuring the absorbance or reflectance of the plurality of samples using the spectrophotometer, and (c) correlating the particle score from step (a) with the measured absorbance or reflectance from step (b).

In another aspect, a method for formulating a feed is provided, the method comprising: (a) calibrating a near infrared reflectance spectrophotometer, comprising: (i) sorting a plurality of forage samples by chop length by passing such samples through a particle separator having a screen and subsequently calculating a particle score for the samples based on the number of samples passing through the screen, (ii) measuring the absorbance or reflectance of the samples using the spectrophotometer, and (iii) correlating the particle score from step (i) with the measured absorbance or reflectance from step (ii), (b) predicting the particle score of a total mixed ration using a near infrared reflectance spectrophotometer correlated according to step (iii), and (c) formulating a feed based on the particle score of the total mixed ration.

### Particle Score

The term “particle score” as used in this disclosure means the percentage of particles of an ingredient (by weight percent) passing through a sieve or screen. The particle score is related to the size of the particle of the ingredient. For example, the size of a forage ingredient can vary depending on the chop length of the forage ingredient. Also for example, the size of a corn ingredient can vary depending on the corn variety, corn moisture, speed of the mill that processed the corn, type of the mill that processed the corn, etc. The particle size of the ingredient may affect the rate and extent of digestibility of the ingredient (e.g., forage) in an animal. For example, adequate forage particle length may assist in proper rumen function. Reduced forage particle size has been shown to decrease the time spent by the animal chewing the forage and cause a trend toward decreased rumen pH in the animal. When cows spend less time chewing, they produce less saliva, which is needed to buffer the rumen of the cow. In comparison, when feed ingredient particles are too long, animals are more likely to sort the ration. This could result in the diet consumed by the animal being very different than the one originally formulated. If rations or forages are too fine, feeding a small amount of long hay or baleage can improve the average ration particle size.

Certain ingredients (e.g., forages) may have a desirable or target particle score. The particle score is inversely related to the size of the particle (i.e., a higher particle score equates to a smaller particle size). For example, as particle score increases, the percentage of neutral detergent fiber (NDF) digestibility increases for ingredients such as forage and more specifically for legume haylage. Also for example, as particle score increases, the net energy of lactation increases for corn silage ingredients and dry corn. Also for example, as particle score increases, the starch digestibility increases for ingredients such as corn, milo, wheat, barley, and oats. Also for example, as particle score increases, the NDF digestibility increases for legume haylage.

### Penn State Particle Separator Method

Particle score of an ingredient may be determined using the Penn State Particle Separator (PSPS) according to the method described in Publication No. DSE 2013-186 published Sep. 26, 2013 by Jud Heinrichs of Penn State, which is hereby incorporated by reference in its entirety. The PSPS provides a tool to quantitatively determine the particle size of forages and total mixed rations (TMR).

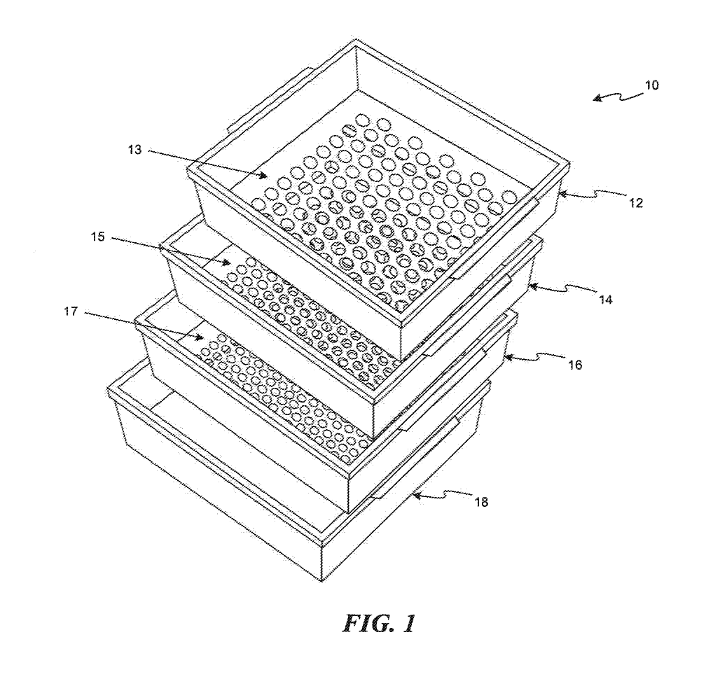

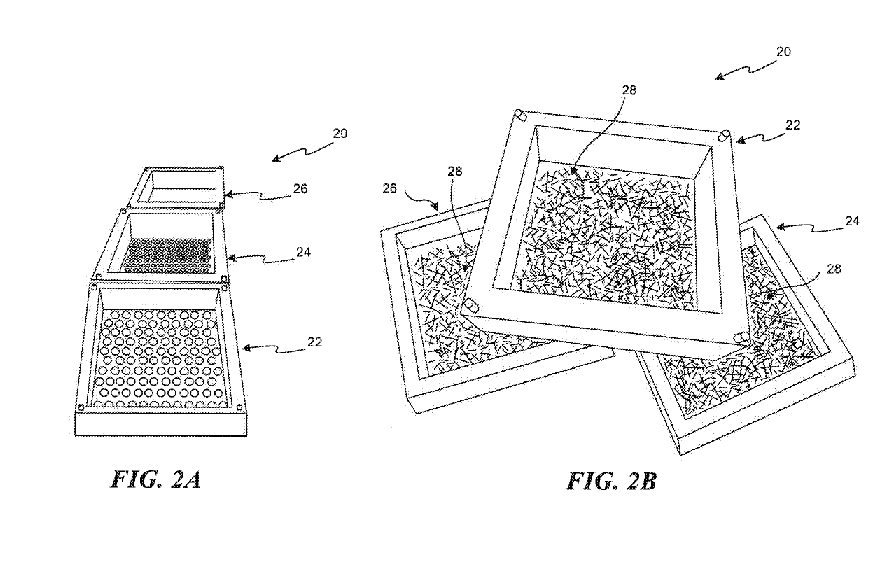

Referring to FIG. 1, a three-sieve PSPS 10 is shown according to an embodiment. As shown in FIG. 1, the three-sieve PSPS has an upper sieve or box 12 having a large diameter screen 13, a middle sieve or box 14 having a medium diameter screen 15, a lower sieve 16 having a smaller diameter screen 17, and a bottom cup or pan 18. Referring to FIG. 2A, a two-sieve PSPS 20 is shown according to an exemplary embodiment. As shown in FIG. 2A, the two-sieve PSPS has an upper sieve or box 22, and a lower sieve 24, and a bottom cup or pan 26. As shown in FIG. 2B, a sample of a plant matter ingredient for a feed ration for an animal is shown as forage 28 having different chop lengths in sieves 22 and 24 and pan 26.

The two-sieve PSPS comprises a sieve having screens with pore sizes through which particles smaller than a certain size can pass, as shown in TABLE 1A.

TABLE 1A

| Screen | Pore Size (inches) | Particle Size (inches) |
|---|---|---|
| Upper Sieve | 0.75 | >0.75 |
| Lower Sieve | 0.31 | 0.31 to 0.75 |
| Bottom Pan | | <0.31 |

The three-sieve PSPS comprises a sieve having screens with pore sizes through which particles smaller than a certain size can pass, as shown in TABLE 1B.

TABLE 1B

| Screen | Pore Size (inches) | Particle Size (inches) |
|---|---|---|
| Upper Sieve | 0.75 | >0.75 |
| Middle Sieve | 0.31 | 0.31 to 0.75 |
| Lower Sieve | 0.16 | 0.16 to 0.31 |
| Bottom Pan | | <0.16 |

To use the three-sieve PSPS, the sieves are stacked on top of each other in the following order: sieve with the largest holes (upper sieve) on top, the medium-sized holes (middle sieve) next, then the smallest holes (lower sieve), and the solid pan on the bottom. Approximately 3 pints of forage or TMR are placed on the upper sieve. Moisture content may cause small effects on sieving properties. Very wet samples (less than forty-five (45) percent dry matter) may not separate accurately. The three-sieve PSPS is designed to describe particle size of the feed offered to the animal. Thus, samples need not be chemically or physically altered from what was fed before sieving. On a flat surface, the sieves are shaken in one direction several times (e.g., five (5) times), and then the separator box is rotated one-quarter turn. This process is repeated several times (e.g., seven times), rotating the separator after each set of, for example, five (5) shakes. The force and frequency of shaking should be great enough to slide particles over the sieve surface, allowing those smaller than the pore size to fall through. It is recommended, although not necessary, to shake the particle separator at a frequency of at least 1.1 Hz (or approximately 1.1 shake per second) with a stroke length of seven (7) in. (or 18 cm). For best results, the frequency of movement is calibrated over a distance of 7 inches for a specified number of times (e.g., 10, 100, 1000 times). The number of full movements divided by time in seconds results in a frequency value that can be compared to the 1.1 Hz recommendation. After shaking is completed, the material is weighed on each sieve and on the bottom pan. See TABLE 2 for data entry and procedures to compute the percentage under each sieve, including an example of the calculation of total weight determined by, for example, a digital scale and cumulative percentages under each sieve. (Where cumulative percentage undersized refers to the proportion of particles smaller than a given size. For example, on average, 95% of feed is smaller than 0.75 inches, 55% of feed is smaller than 0.31 inches and 35% of feed is smaller than 0.16 inches.)

TABLE 2
Record and Calculate Data

| | | Proportion |
|---|---|---|
| | Weight | Remaining |
| Sample | Retained | On Each Sieve |
| Upper sieve | 10 grams [a] | a/e * 100 = 10/200 * 100 = 5% |
| (0.75 inches) | | |
| Middle sieve | 80 grams [b] | b/e * 100 = 80/200 * 100 = 40% |
| (0.31 inches) | | |
| Lower sieve | 40 grams [c] | c/e * 100 = 40/200 * 100 = 20% |
| (0.16 inches) | | |
| Bottom pan | 70 grams [d] | d/e * 100 = 70/200 * 100 = 35% |
| (<0.16 inches) | | |
| Sum of Weights | 200 grams [e] | |

Compute Cumulative Percentage Undersized

| | | |
|---|---|---|
| % Under | f = 100 − (a/e * 100) | 100 − 5 = 95% undersized |
| upper sieve | | |
| % Under | g = f − (b/e * 100) | 95 − 40 = 55% undersized |
| middle sieve | | |
| % Under | h = g − (c/e * 100) | 55 − 20 = 35% undersized |
| lower sieve | | |

To use the two-sieve PSPS, the procedure is substantially the same as the one describe above for using the three-sieve PSPS except that the sieve having a screen size of 0.31 inches is not used.

### Alternative Particle Score Methods

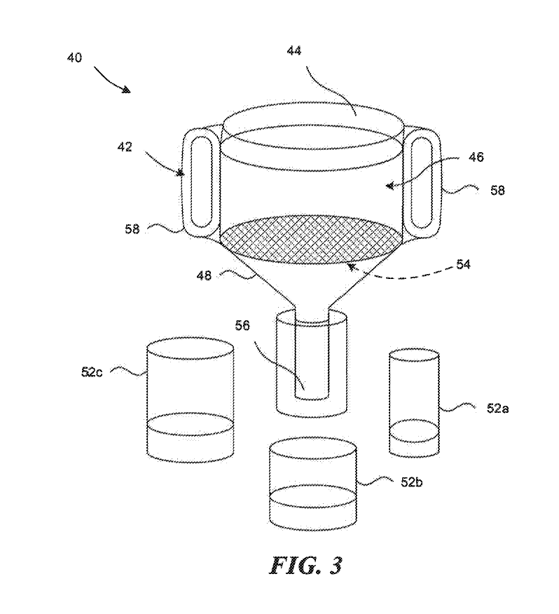

Particle score may also be determined using the Alternative Particle Scorer (APS). The APS provides a tool to quantitatively determine the particle size of, for example, corn forages. An APS 40 is shown in FIG. 3 having a shaker 42 with a housing 44 having a large diameter body 46 for intake of a sample of corn forage, and a small diameter body 48 for retention of the sample, which may be measured in a grain cup 52a, cup 52b or cup 52c. Housing 44 is shown with a screen 54 through which the sample is provided to a reservoir (shown as a bottom pan 56). Using handles 58 on top of housing 44 allows for shaking of the sample, especially when APS 40 is shaken on a flat surface (such as the ground or floor) so that the sample is passed through screen 54. One portion of the sample having a larger particle size is retained on screen 54, and another portion of the sample having a smaller particle size is retained on the bottom pan or grain receptacle 56. The pore size of the screen and size of the particles that pass through the screen are shown in TABLE 3.

TABLE 3

| Screen | Pore Size (inches) | Particle Size (inches) |
|---|---|---|
| Sieve | 0.065 | >0.065 |
| Bottom Pan | | <0.065 |

In order to determine particle score, the following procedure may be used for corn forage run through the APS. The appropriately sized cup (depending on the ingredient of interest) is fastened into the grain receptacle in the smaller diameter end of the shaker body. The screen is placed into the larger diameter end of the shaker body. The grain sample cup is filled one-half full with a representative sample of corn forage. (Note, to ensure consistent readings the sample level can be read parallel to the operator's eye level.) The cup is covered with the palm of the operator's hand and tapped (e.g., five times). The grain sample cup is then topped off with additional grain sample, covered with the palm of the operator's hand, and tapped (e.g., five more times) so that the grain sample cup is approximately three-fourths full. The remainder of the gain sample cup is then filled with additional sample, and leveled off the top (e.g., with the operator's finger), so the top of the sample is level with the top of the grain sample cup. The sample is then poured from the grain sample cup into the larger diameter body having the screen. The APS is kept parallel to the ground and shaken vigorously for thirty seconds. The screen is gently removed and observed for any sample hanging on the sides of the larger diameter body. (If any sample is hung up on the sides of the housing, the sides are gently tapped on a firm surface until all sample is captured on the screen.) The grain sample cup is then removed and covered with the palm of the operator's hand. Readings are recorded for the weights of the sample retained on the screen and those retained in the cup. Note, if high moisture and dry ingredients are being sieved consecutively, it is advantageous to run the dry ingredients first (so the dry ingredient does not adhere to residual moisture left from the previous sample).

According to another alternative embodiment, particle size may be determined by the American Society of Agricultural and Biological Engineers' (ASABE) standard for particle size analysis and distribution, which is hereby incorporated by reference in its entirety.

### NIRs Generally

The terms “near infrared” (“NIR”) and “near infrared spectroscopy” (“NIRs”) as used in this disclosure relate to a spectroscopy analyzing method based on the excitation of molecular vibrations with electromagnetic radiation in the near infrared wavelength region. The near infrared wavelength region (i.e., 800 nm-2500 nm) lies between visible light wavelength region (380 nm-800 nm) and mid-infrared radiation wavelength region (2500 nm-25000 nm). NIRs measures the intensity of the absorption of near infrared light by a substance or mixture (such as plant matter). NIRs detects overtones and combination of molecules' fundamental vibrations in the substances (e.g., plant matter) containing CH—, OH— and NH— groups (e.g., fats, proteins carbohydrates, organic acids, alcohol, water, etc.). As used in this disclosure, the term “spectroscopy” can refer to all molecular spectroscopy, including near infrared reflectance spectroscopy, near infrared transmission spectroscopy, ultra violet and visible spectroscopy, Fourier transform near infrared spectroscopy, Raman spectroscopy, and mid-infrared spectroscopy.

Operation of the NIR device or instrument includes the provision of a beam of light to the sample (e.g., dry plant matter). The light that is reflected or transmitted by the sample is collected as information (i.e., spectra). (An NIR instrument may be run in reflection mode, transmission mode, transflection mode, etc.) More specifically, the software of the NIR instrument measures the amount of energy returned to detectors from the sample, which is subtracted from a reference spectrum, and the resulting absorbance spectrum is plotted. An NIR spectrum consists of a number of absorption bands that vary in intensity due to energy absorption by specific functional groups in the sample. Based on Beer's law, the absorption is proportional to the concentration of a chemical (or physical) component in the sample, thus the spectra information is utilized to quantify the chemical (or physical) composition of biological materials (e.g., plant matter).

The use of NIR to measure parameters of interest has several advantages over wet chemistry, such as non-destructive, non-invasive measurement with little or no sample preparation, nearly instantaneous measurement, and fast response times (e.g., real time, scan completed within 1 minute, etc.), easy and reliable operation, ability to test for multiple nutrients simultaneously through one scan (e.g., moisture, crude protein, fat, ash, fiber, etc.), long-term calibration stability allows direct calibration transfer between similar NIR instruments and indirect calibration transfer between different instrument platforms, low cost operational cost, quick and easy implementation and maintenance, reliability with improved precision and consistency, etc. Further, NIR instruments may be used in the lab and may be portable for use in the field and on the farm.

### NIRs Calibrations

The term “NIR or NIRs calibration” as used in this disclosure means a mathematical model that correlates NIR spectra to a reference or standard (e.g., wet chemistry value). NIRs involves the calibration (or association) of NIR spectra against a primary method or direct measurement of a sample (also referred to as “wet chemistry”). Examples of primary methods of direct measurement using wet chemistry include a protein analysis by the Kjeldahl or Leco protein analyzer, fiber analysis by the Ankom Fiber Analyzer, animal digestion such as digested neutral detergent fiber (dNDF), and invitro protein digestibility (IVpd) measured by invitro techniques.

In some embodiments, in order to create a calibration, the following steps can be conducted: 1) Construct a database comprising wet chemistry values and NIR spectra or values, 2) Develop a mathematical model (e.g., NIR calibration); 3) Verify the mathematical model using independent samples not included in the original database; 4) Run or scan new samples on an NIR instrument using the mathematical model to predict wet chemistry values; and 5) Validate the mathematical model.

1. Construct Database. To construct the database, a number of representative samples are collected to cover expected variations. Each sample has two areas of interest: (i) the reference values of the sample derived from a primary method of direct measurement (also referred to as “wet chemistry” or “lab value”); and (ii) the spectra derived from running the samples in the NIR instrument. This dataset is also referred to as a training data set.

2. Develop Mathematical Model. The wet chemistry measurements from the training data set are used as reference data and NIR spectra from the training data set are regressed on the wet chemistry data in model development. To develop a mathematical model (or equation or NIR calibration), chemometric technics are used. The term “chemometrics” as used in this disclosure means the science of extracting information from chemical systems by data-driven means. More specifically, multivariate calibration methods are used to yield the best fit of the NIR spectra to the reference value (e.g., training data set), resulting in the NIR calibration models (which predict or correspond to the properties of interest). In other words, a model (or calibration) is developed which can be used to predict properties of interest based on measured properties of the chemical system (e.g., NIR spectra), such as the development of a multivariate model relating the multi-wavelength NIR spectral response to analyte concentration in the sample. Various calibration algorithms are available in chemometric software to develop the calibration model, such as MLR (multiple linear regressions), MPLS (modified partial least squares regression), PCA (principal component analysis), ANN (artificial neural network), local calibration, etc. Other multivariate calibration techniques include, for example partial-least squares regression, principal component regression, local regressions, neural networks, support vector machines (or other methods).

3. Verify the Mathematical Model. A testing set serves as an independent set (i.e., different from the calibration training data set) to verify the calibration model performance. The testing set includes plotting the wet chemistry values of the sample against the mathematical model that has been developed.

4. Scan Samples. New samples are then scanned on the NIR instrument using the mathematical model that has been developed to predict the wet chemistry values of the new samples. The resulting spectra patterns for these new samples are correlated to the reference measurements using the NIR calibration model previously created. Predictions are thus generated for the intended parameter of interest.

5. Validate Mathematical Model. The NIR calibration is then “validated.” A good NIR calibration demonstrates a high correlation between NIR predicted values and the reference (or wet chemistry) values. Validation includes a process similar to creating the calibration, but accounts for instrument specific bias. Therefore, the final NIR calibration is bias-corrected. It includes the original NIR calibration and accounts for the bias of the specific individual NIR instrument.

### Formulating a Feed for Animals

The NIRs calibration for particle score may be developed for plant, animal, or mineral ingredients. Examples of plant matter ingredients include protein ingredients, grain products, grain by-products, roughage products, fats, minerals, vitamins, additives or other ingredients according to an exemplary embodiment. Protein ingredients may include, for example, animal-derived proteins such as: dried blood meal, meat meal, meat and bone meal, poultry by-product meal, hydrolyzed feather meal, hydrolyzed hair, hydrolyzed leather meal, etc. Protein ingredients may also include, for example, marine products such as: fish meal, crab meal, shrimp meal, condensed fish soluble, fish protein concentrate, etc. Protein ingredients may also further include, for example, plant products such as: algae meal, beans, coconut meal, cottonseed meal, rapeseed meal, canola meal, linseed meal, peanut meal, soybean meal, sunflower meal, peas, soy protein concentrate, dried yeast, active dried yeast, etc. Protein ingredients may include, for example, milk products such as: dried skim milk, condensed skim milk, dried whey, condensed whey, dried hydrolyzed whey, casein, dried whole milk, dried milk protein, dried hydrolyzed casein, etc. Grain product ingredients may include, for example, corn, milo, oats, rice, rye, wheat, etc. Grain by-product ingredients may include, for example, corn bran, peanut skins, rice bran, brewers dried gains, distillers dried grains, distillers dried grains with soluble, corn gluten feed, corn gluten meal, corn germ meal, flour, oat groats, hominy feed, corn flour, soy flour, malt sprouts, rye middlings, wheat middlings, wheat mill run, wheat shorts, wheat red dog, feeding oat meal, etc. Roughage product ingredients may include, for example, corn cob fractions, barley hulls, barley mill product, malt hulls, cottonseed hulls, almond hulls, sunflower hulls, oat hulls, peanut hulls, rice mill byproduct, bagasse, soybean hulls, soybean mill feed, dried citrus pulp, dried citrus meal, dried apple pomace, dried tomato pomace, ground straw, etc. Mineral product ingredients may include, for example, ammonium sulfate, basic copper chloride, bone ash, bone meal, calcium carbonate, calcium chloride, calcium hydroxide, calcium sulfate, cobalt chloride, cobalt sulfate, cobalt oxide, copper sulfate, iron oxide, magnesium oxide, magnesium sulfate, manganese carbonate, manganese sulfate, dicalcium phosphate, phosphate deflourinated, rock phosphate, sodium chloride, sodium bicarbonate, sodium sesquincarbonate, sulfur, zinc oxide, zinc carbonate, selenium, etc. Vitamin product ingredients may include, for example, vitamin A supplement, vitamin A oil, vitamin D, vitamin B 12 supplement, vitamin E supplement, riboflavin, vitamin D3 supplement, niacin, betaine, choline chloride, tocopherol, inositol, etc. Additive product ingredients may include, for example, growth promoters, medicinal substances, buffers, antioxidants, preservatives, pellet-binding agents, direct-fed microbials, etc.

According to a preferred embodiment, the NIRs calibrations are developed for forage ingredients. Forage is plant material (mainly plant leaves and stems) eaten by grazing livestock. The term “forage” as used in this disclosure, includes plants cut for fodder and carried to the animals, such as hay or silage. Grass forages include, for example, bentgrasses, sand bluestem, false oat-grass, Australian bluestem, hurricane grass, Surinam grass, koronivia grass, bromegrasses, buffelgrass, Rhodes grass, orchard grass bermudagrass, fescues, black spear grass, West Indian marsh grass, jaragua, southern cutgrass, ryegrasses, Guinea grass, molasses grass, dallisgrass, reed canarygrass, timothy, bluegrasses, meadow-grasses, African bristlegrass, kangaroo grass, intermediate wheatgrass, sugarcane, etc. Herbaceous legume forages include, for example, pinto peanut, roundleaf sensitive pea, butterfly-pea, bird's-foot trefoil, purple bush-bean, burgundy bean, medics, alfalfa, lucerne, barrel medic, sweet clovers, perennial soybean, common sainfoin, stylo, clovers, vetches, creeping vigna, etc. Tree legume forages include, for example, mulga, silk trees, Belmont siris, lebbeck, leadtree, etc. Silage forages include, for example, alfalfa, maize (corn), grass-legume mix, sorghums, oats, etc. Forage may include “haylage.” The term haylage as used in this disclosure means silage made from grass that has been partially dried. Crop residues used as forage include, for example, sorghum, corn or soybean stover, etc. Other examples of forages include, for example, corn silage, brown midrib corn silage, sugarcane silage, barley silage, haylage grass, haylage legume, haylage mixed, haylage small grain, haylage sorghum sudan, fresh grass, fresh legume, fresh mixed, fresh small grain, hay grass, hay legume, hay mixed, hay small grain and straw, high moisture shelled corn, high moisture ear corn, total mixed ration, etc.

The NIR calibration for particle score may be used to determine nutritive properties of ingredients, which may be used to further formulate an animal feed. For example, forage samples may be gathered from a farm and transported to a laboratory or other analytical facility. The forage sample as received (i.e., not further dried or ground) may be scanned using an NIR device. The NIR output may be used to predict a particle score value using NIR calibration methods of the present disclosure. The particle score value for the forage ingredient may be transferred to animal prediction software or feed ration balancer software, such as for example, MAX software, available from Cargill, Incorporated, Wayzata, Minn., USA, along with nutrient information for the same forage, which may include, for example, protein information, moisture information, fat information, etc., to determine, for example, the digestibility of the forage. If the forage is deemed to have a sub-optimal particle score, then additional nutrients (e.g., additional forages) may be included in the diet to account for the lack of digestibility of the forage. The term “animal feed” as used in this disclosure means a feed ration and/or supplement produced for consumption by an animal. The term “animals” as used in this disclosure include, for example, bovine, porcine, equine, caprine, ovine, avian animals, seafood (aquaculture) animals, etc. Bovine animals include, but are not limited to, buffalo, bison, and all cattle, including steers, heifers, cows, and bulls. Porcine animals include, but are not limited to, feeder pigs and breeding pigs, including sows, gilts, barrows, and boars. Equine animals include, but are not limited to, horses. Caprine animals include, but are not limited to, goats, including does, bucks, wethers, and kids. Ovine animals include, but are not limited to, sheep, including ewes, rams, wethers, and lambs. Avian animals include, but are not limited to, birds, including chickens, turkeys, and ostriches (and also include domesticated birds also referred to as poultry). Seafood animals (including from salt water and freshwater sources) include, but are not limited to, fish and shellfish (such as clams, scallops, shrimp, crabs and lobster). The term “animals” as used in this disclosure also includes ruminant and monogastric animals. As used in this disclosure, the term “ruminant” means any mammal that digests plant-based ingredients using a regurgitating method associated with the mammal's first stomach or rumen. Such ruminant mammals include, but are not limited to, cattle, goats, sheep, giraffes, bison, yaks, water buffalo, deer, camels, alpacas, llamas, wildebeest, antelopes and pronghorns. The term “animals” as used in this disclosure also includes domesticated animals (e.g., dogs, cats, rabbits, etc.), and wildlife (e.g., deer).

The formulation of the animal feed may be a compound feed, a complete feed, a concentrate feed, a premix, and a base mix according to alternative embodiments. The term “compound feed” as used in this disclosure means an animal feed blended to include two or more ingredients that assist in meeting all the daily nutritional requirements of an animal. The term “complete feed” as used in this disclosure means an animal feed that is a complete feed, i.e., a nutritionally balanced blend of ingredients designed as the sole ration to provide all the daily nutritional requirements of an animal to maintain life and promote production without any additional substances being consumed except for water. The term “concentrate teed” as used in this disclosure means an animal feed that includes a protein source blended with supplements or additives (e.g., vitamins, trace minerals, other micro ingredients, macro minerals, etc.) to provide a part of the ration for the animal. The concentrate feed may be fed along with other ingredients (e.g., forages in ruminants). As used in this disclosure, the term “premix” means a blend of primarily vitamins and trace minerals along with appropriate carriers in an amount of less than about five percent (5.0%) inclusion per ton of complete feed. The term “base mix” as used in this disclosure means a blend containing vitamins, trace minerals and other micro ingredients plus macro minerals such as calcium, phosphorus, sodium, magnesium and potassium, or vitamin or trace mineral in an amount of less than ten percent (10.0%) inclusion per ton of complete feed.

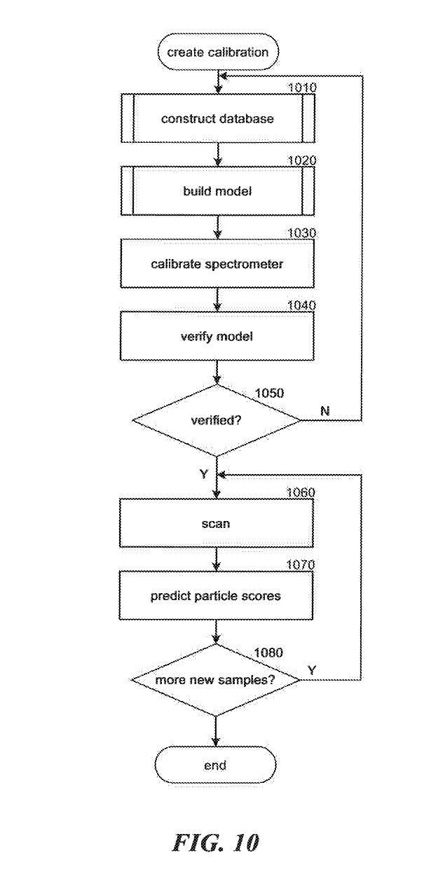

FIG. 10 is a flow diagram illustrating the processing of a create calibration component in accordance with some embodiments of the disclosed technology. In block 1010, the component invokes a construct database component to build a database of sample data by analyzing data associated with a number of representative collected samples. For example, the component may analyze several variations of feed and forage compositions to establish a comprehensive database of sample data. The sample data may include, for each sample, reference particle score values of the sample derived from a primary method of direct measurement (also referred to as “wet chemistry” or “lab value”), spectra information (e.g., spectra patterns) derived from scanning the samples in the NIR instrument, and so on. In block 1020, the component builds a representative model of the sample data by correlating a portion of the spectra data from the sample database to the corresponding reference values from the sample database. For example, the component may generate a multivariate linear regression correlating spectra data to reference particle score values using 75% of the data from the sample database. The representative model provides for the prediction of a particle score or scores based on spectra information. As discussed above, one of ordinary skill in the art will recognize that any one or more algorithms for correlating values can be used, such as MLR (multiple linear regressions), MPLS (modified partial least squares regression), PCA (principal component analysis), ANN (artificial neural network), local calibration, etc. Other multivariate calibration techniques include, for example partial-least squares regression, principal component regression, local regressions, neural networks, support vector machines, and so on. In block 1030, the component calibrates the spectrometer using the constructed model by, for example, loading model values into the spectrometer. In block 1040, the component verifies the model by testing sample data from the database not used to generate the model (e.g., the 25% of the data not used in the example above). For example, the component uses the model and spectra values for “verification samples” in the sample database to “predict” particle scores for these “verification samples” and compares these “predicted” values to the actual particle score values in the database. If the average difference between the predicted and actual values is within a predetermined range, then the model can be verified. In block 1050, if the model is verified then the component continues at block 1060, else the component loops back to block 1010 to reconstruct a database. In block 1060, the component collects spectra information from a spectrometer for a new sample. In block 1070, the component uses the model to correlate the collected spectra information for the new sample to reference particle score values to predict particle score(s) for the sample. These predicted particle score values can be used to determine whether a particular feed composition is suitable for a particular purposed or needs to be modified. In decision block 1080, if there are additional new samples then the component loops back to block 1060 to collect spectra information for the new sample, else the component completes.

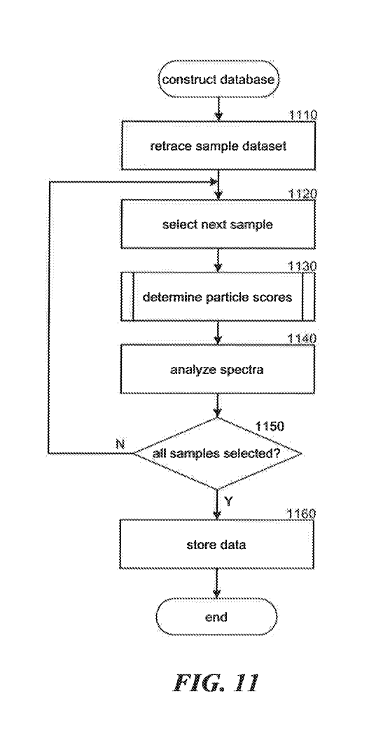

FIG. 11 is a flow diagram illustrating the processing of a construct database component in accordance with some embodiments of the disclosed technology. In block 1110, the component retrieves a sample dataset. For example, the component may retrieve previously generated sample data from a database containing, for each sample, information about how the samples were processed (e.g., sieve type, screen/pore sizes, number of screens, weight information, spectra information). In blocks 1120-1150, the component loops through each of a plurality of samples to process each sample. In block 1120, the component selects the next sample. In block 1130, the component invokes a determine particle scores component for the sample. In block 1140, the component retrieves spectra information for the sample. In decision block 1150, if all of the samples have been selected then the component continues at block 1160, else the component loops back to block 1120 to select the next sample. In block 1160, the component stores the determined particle scores and spectra information in a database and then completes. In some embodiments, the determined particles scores and spectra information may be stored as separate individual values or may be stored as a composite or vector of values.

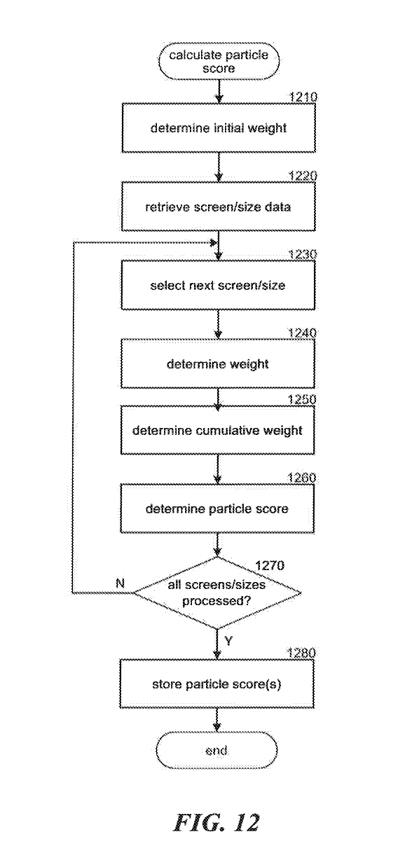

FIG. 12 is a flow diagram illustrating the processing of a determine particle scores component in accordance with some embodiments of the disclosed technology. The component is invoked to generate a particle score or scores for a sample. In block 1210, the component determines a weight for the sample by, for example, receiving an indication of the weight from a digital scale or retrieving the weight from a data source. In block 1220, the component retrieves screen/size data for each screen through which the sample is processed, such as the number of screens in a sieve used to process the sample and the pore size of each screen. In block 1230-1270, the component loops through each screen to process the sample and generate particle scores for each screen. In block 1230, the component selects the next screen (or bottom pan), starting with the bottom pan and moving up through each screen. In block 1210, the component determines the weight of the sample retained by the screen (or bottom pan). In block 1250, the component determines the cumulative weight of the material in or below the screen (or bottom pan). In block 1260, the component determines the particle score for the screen (or bottom pan) based on the weight of the sample collected by the screen (or bottom pan) and the combined weight of the samples retained by all of the screens and the bottom pan. In decision block 1270, if all of the screens have been processed, then the component continues at block 1280, else the component continues at block 1280. In block 1280, the component stores the determined particle scores for each screen (or bottom pan) in association with the sample. In some embodiments, the determined particles scores may be stored as separate individual values or may be stored as a composite or vector of values. For example, each sample may have a unique identifier that is stored in association with the data.

### EXAMPLES

Aspects of certain methods in accordance with aspects of the invention are illustrated in the following example.

### Example 1

A near infrared spectroscopy calibration for particle score of a plant matter ingredient (e.g., forage) was built by: 1) Constructing a database comprising wet chemistry values and NIR spectra values; 2) Developing a mathematical model (e.g., NIR calibration); 3) Verifying the mathematical model using independent samples not included in the original database; 4) Running or scanning new samples on an NIR instrument using the mathematical model to predict wet chemistry values; and 5) Validating the mathematical model. The mathematical model (e.g., NIR calibration) is useful for predicting the particle score of ingredients such as plant matter ingredients, such as forages.

Materials and Instrument. In this example, wet forages were received at the lab from the field or bunk on a daily basis. A wet, unground forage sample was filled in a large cup with quartz glass and scanned on FOSS DS2500 NIR instrument. The spectrum was thus acquired via FOSS ISISCAN Nova operation software with wavelengths ranging from 400 to 2500 nm. The forage products covered in this example include 19 different forage types, e.g., haylage grass/legume/mixed/sorghum sudan/small grains, fresh grass/legume/mixed/small grains, hay grass/legume/mixed/small grain straw, total mixed ration (TMR) and high moisture ear corn/shelled corn.

Reference Methods. In this example, the Alternative Particle Scorer (APS) was used to quantify forage particle size by measuring the mass of a wet forage sample passing through a brass screen with the 0.065 inch diameter. A two-sieve Penn State Particle Separator (PSPS) was used to obtain different particle size fractions with top (longer than 0.75 inches), middle (between 0.31 and 0.75 inches) and bottom (shorter than 0.31 inches). Only TMR samples were tested on Penn State particle method. All the particle score results were reported in sample mass percentage.

Mathematical Model Development. In this example, a database was established in the lab comprising collected spectra along with corresponding reference (wet chemistry) particle score values. The database was split into a calibration training set and a testing set (i.e., verification set). The calibration training set (about 80% of data) was employed to train a calibration model, while the testing set (around 20%) was utilized to examine the model performance on an independent dataset. Spectra analysis and model development were performed using FOSS WINISI 4 chemometrics software. A calibration technique, e.g., modified partial least squares (MPLS) with cross validation, was chosen to develop the models for these small databases. In order to minimize the impact of spectral artifacts and avoid model over-fitting, the spectra were evaluated first by identifying and removing noisy wavelength regions. Model optimization was conducted by applying and examining various spectral transformation techniques and spectral pretreatment methods.

Mathematical Model Validation. In this example, mathematical model (e.g., NIR calibration) performance was evaluated by using calibration and validation statistical parameters, such as: (i) SEPc (standard calibration prediction error); (ii) Slope (correlation between reference values and NIR predictions); (iii) R2 (coefficient of determination); and (iv) RPD (relative prediction deviation, ratio of population StdDev (standard deviation) of reference values to SEPc). The mathematical model performance was evaluated on the calibration database itself in the first place. The optimum calibration parameters such as the factors, spectral preprocessing techniques were determined by the performance statistics of cross-validation during calibration model development. Then the model performance was verified and examined in independent testing (external validation).

TABLE 4 shows a comparison between NIR predicted particle scores with actual particle scores (according to the Alternative Particle Scorer method). The validated range of particle size (min and max values) per ingredient is also listed in TABLE 4. Additionally, the population standard deviation for both wet chemistry and NIR results are illustrated in TABLE 4 to show the population variability existing in the two data sets. The ‘No. of samples’ refer to the number of samples used in the testing sets.

TABLE 4

| | | | Average of | | | | StdDev of | | |
|---|---|---|---|---|---|---|---|---|---|
| | | Average of | predicated | | Min of | Max of | reference | StdDev of | |
| | | actual | by NIR | Average of | actual | actual | method | predicted | |
| | | particle | particle | Residual | particle | particle | (i.e., actual | by NIR | |
| | | score | score | (Actual − | score | score | particle | particle | No. of |
| Abbreviation | Ingredient | (%) | (%) | Predicated) | (%) | (%) | score) | score | samples |
| BMR | Brown Midrib | 27.18 | 27.18 | 0.00 | 14.50 | 48.00 | 9.08 | 3.12 | 29 |
| | Corn Silage | | | | | | | | |
| CS | Corn silage | 28.34 | 28.34 | 0.00 | 18.50 | 43.50 | 7.48 | 4.26 | 15 |
| EHB | Barley Silage | 26.45 | 26.52 | −0.08 | 17.00 | 37.00 | 8.07 | 3.31 | 6 |
| EHFG | Fresh Grass | 23.39 | 22.44 | 0.95 | 15.00 | 35.33 | 7.89 | 5.20 | 6 |
| EHFL | Fresh Legume | 26.44 | 26.44 | 0.00 | 2.00 | 36.67 | 8.53 | 5.35 | 13 |
| EHFM | Fresh Mixed | 18.08 | 19.50 | −1.42 | 9.00 | 23.00 | 6.62 | 3.33 | 4 |
| EHFSG | Fresh Small | 15.67 | 14.13 | 1.53 | 2.00 | 29.33 | 19.33 | 2.20 | 2 |
| | Grain | | | | | | | | |
| EHG | Haylage Grass | 28.10 | 28.10 | 0.00 | 6.33 | 58.33 | 16.11 | 3.85 | 16 |
| EHL | Haylage | 32.65 | 32.65 | 0.00 | 19.67 | 48.00 | 8.46 | 4.18 | 14 |
| | Legume | | | | | | | | |
| EHM | Haylage | 25.33 | 25.33 | 0.00 | 14.33 | 39.00 | 6.71 | 2.59 | 17 |
| | Mixed | | | | | | | | |
| EHSG | Haylage Small | 25.75 | 26.00 | −0.26 | 9.00 | 34.33 | 7.40 | 5.20 | 12 |
| | Grain | | | | | | | | |
| EHSS | Haylage | 21.52 | 21.52 | 0.00 | 2.00 | 41.67 | 9.04 | 6.27 | 28 |
| | Sorghum | | | | | | | | |
| | Sudan | | | | | | | | |
| HG | Hay Grass | 39.75 | 39.75 | 0.00 | 28.00 | 57.00 | 11.43 | 8.63 | 10 |
| HL | Hay Legume | 59.18 | 59.18 | 0.00 | 27.33 | 75.00 | 15.12 | 10.53 | 9 |
| HM | Hay Mixed | 58.46 | 58.46 | 0.00 | 27.67 | 80.33 | 13.02 | 11.80 | 15 |
| HMEC | High Moisture | 19.80 | 19.80 | 0.00 | 9.67 | 34.00 | 6.92 | 4.78 | 23 |
| | Ear Corn | | | | | | | | |
| HMSC | High Moisture | 39.33 | 39.33 | 0.00 | 15.33 | 67.33 | 18.03 | 6.58 | 20 |
| | Shelled Corn | | | | | | | | |
| HSG | Hay Small | 37.88 | 37.88 | 0.00 | 23.67 | 57.00 | 8.97 | 7.80 | 19 |
| | Grain and | | | | | | | | |
| | Straw | | | | | | | | |
| TMR | Total Mixed | 43.70 | 43.70 | 0.00 | 15.67 | 86.00 | 20.26 | 14.63 | 46 |
| | Ration | | | | | | | | |

It can be seen from TABLE 4 that the average residual (average difference between actual and NIR predicted) is relatively negligible, which means that NIR estimation is comparable to the wet chemistry method.

TABLE 5 shows a comparison between NIR predicted particle scores with actual particle scores (according to the Penn State Particle Separator method) for a total mixed ration (TMR).

TABLE 5

| | | Average of | | | | StdDev of | StdDev of | |
|---|---|---|---|---|---|---|---|---|
| | Average of | predicated | | Min of | Max of | reference | NIR | |
| | actual | by NIR | Average of | actual | actual | method | predicted | |
| | particle | particle | Residual | particle | particle | (i.e., actual | by NIR | |
| | score | score | (Actual − | score | score | particle | particle | No. of |
| Product | (%) | (%) | Predicated) | (%) | (%) | score) | score | samples |
| TMR-BOTTOM | 20.74 | 20.73 | 0.01 | 3.50 | 56.67 | 12.63 | 10.31 | 61 |
| TMR-MIDDLE | 20.99 | 20.99 | 0.00 | 4.29 | 69.57 | 19.07 | 17.62 | 50 |
| TMR-TOP | 57.20 | 57.20 | 0.00 | 0.87 | 94.04 | 31.04 | 24.96 | 50 |

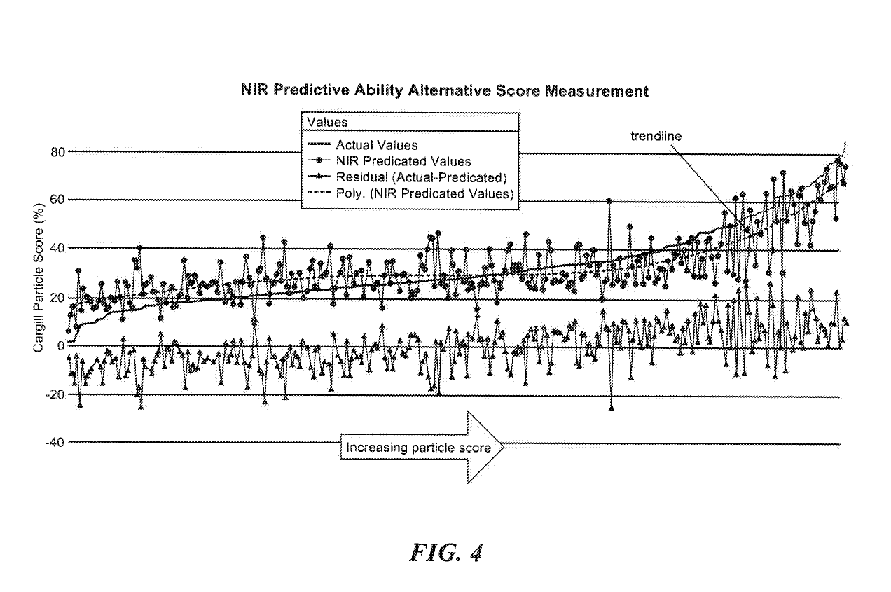

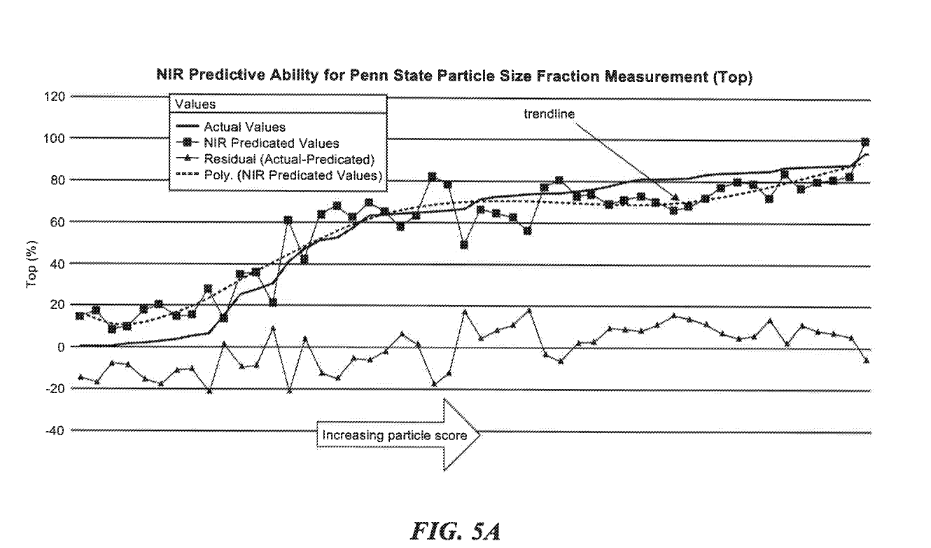

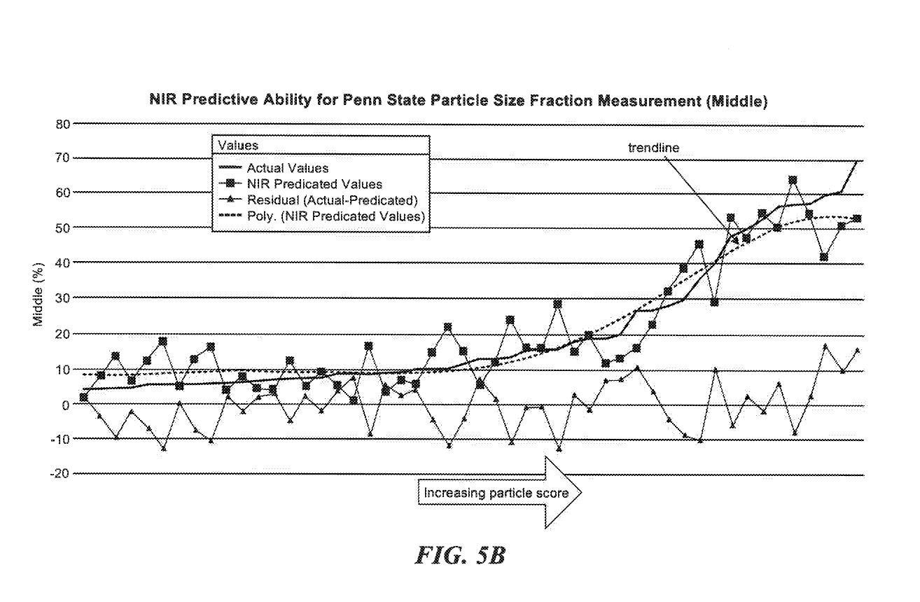

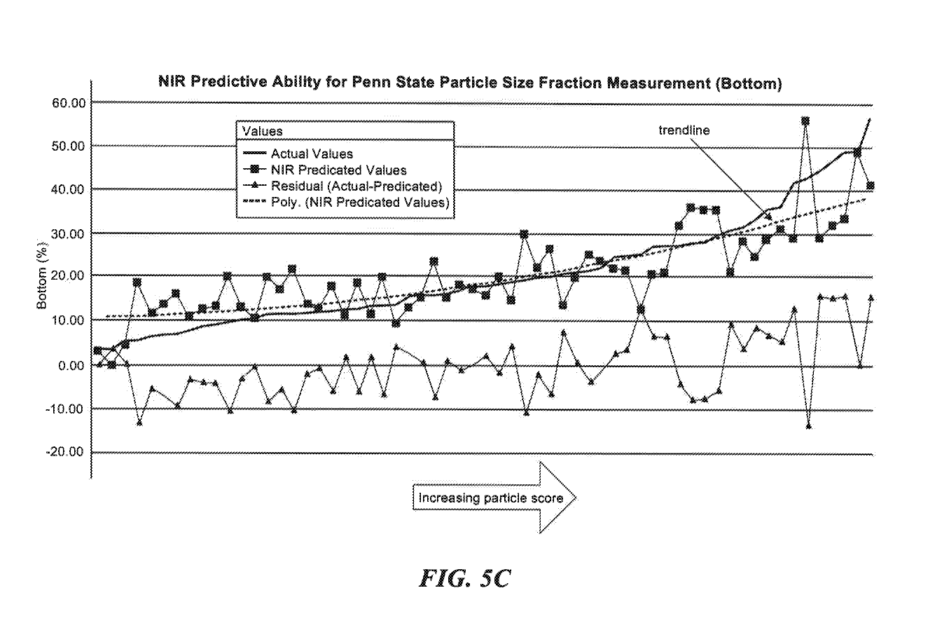

The graphs of FIG. 4 and FIGS. 5A through 5C illustrate the comparability of NIR results and wet chemistry measurements from testing (external validation) set for the 19 different forage ingredients as described in TABLE 4. The X-axis of FIG. 4 and FIGS. 5A through 5C represents testing samples sorted on the particle score from low to high (increasing from left to right), while the Y-axis denotes particle score in the percentage of sample mass. The graphs of FIG. 4 and FIGS. 5A through 5C were generated to help analyze and evaluate NIR model predictability across the range of particle scores and also serve as a guideline for future calibration model improvement. The wet chemistry and NIR results are coded in the graphs of FIG. 4 and FIGS. 5A through 5C along with trend line and the pattern of residual (difference between wet chemistry and NIR results) across the particle score range.

FIG. 4 shows NIR predictive ability across Alternative Particle Scorer range from 2% to 86%. From the trendline and residuals indicated in the FIG. 4, it appears that the NIR calibration overestimates Alternative Particle Scorer in the low values and underestimates it in the high values. The NIR calibration may be further optimized by collecting more samples especially in the low and high values and using various calibration techniques (ANN and MPLS or local).

FIG. 5A shows NIR predictive ability across Penn State Particle Size Fraction range (Top Sieve) from 0.8% to 94.0%.

FIG. 5B shows NIR predictive ability across Penn State Particle Size Fraction range (Middle Sieve) from 4.3% to 69.6%.

FIG. 5C shows NIR predictive ability across Penn State Particle Size Fraction range (Bottom Sieve) from 3.5% to 56.7%.

As seen in FIGS. 5A through 5C, from top, middle to bottom sieve, the NIR prediction accuracy increases with less pronounced trending in low and high values. It is also observed that the fluctuation range of residual (difference between actual and predicted value) drops in middle and bottom sieve measurements compared to top measurement. The improvement in NIR model performance may be contributed to by the filtering out of the large particles in the top sieve, which implies that NIR offers a better predictability with more uniform particle size distribution.

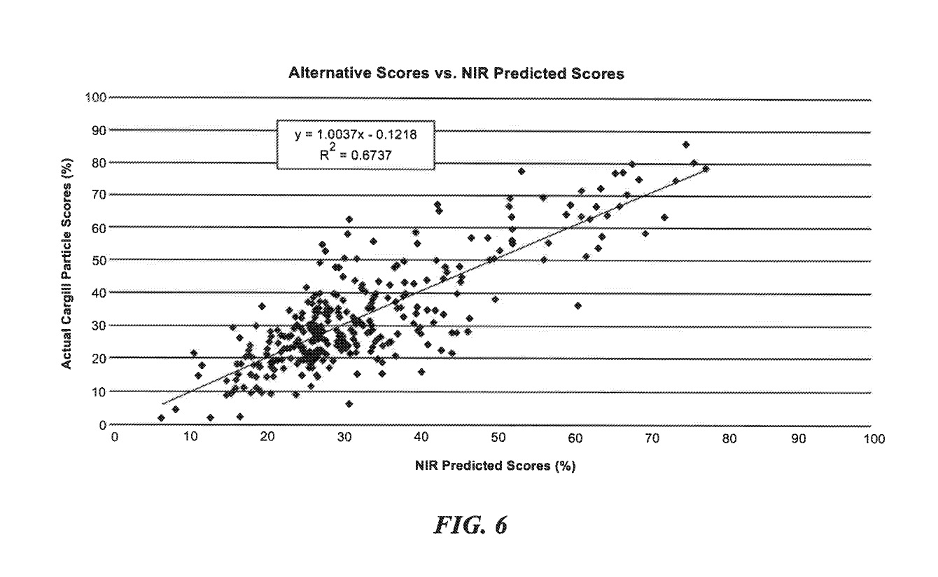

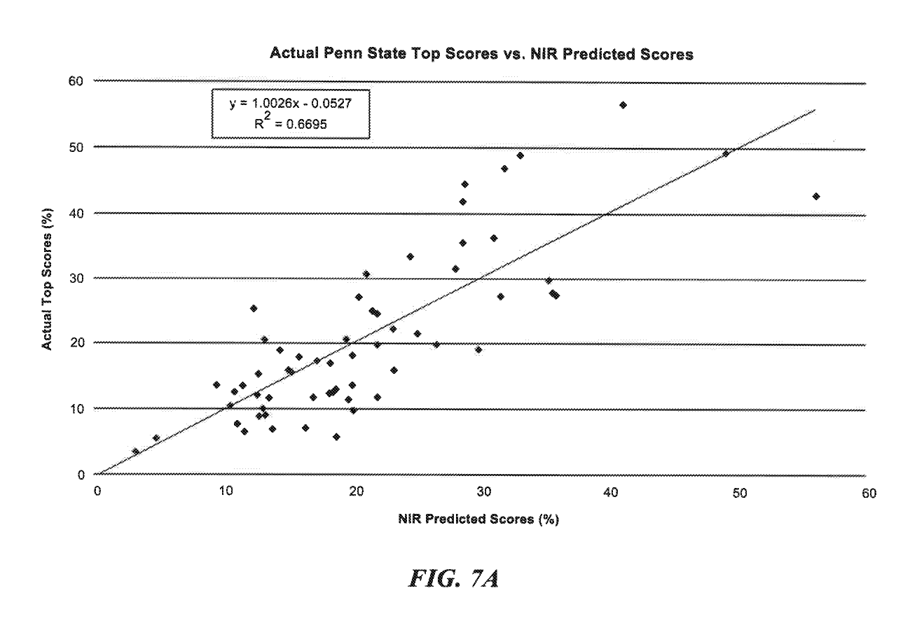

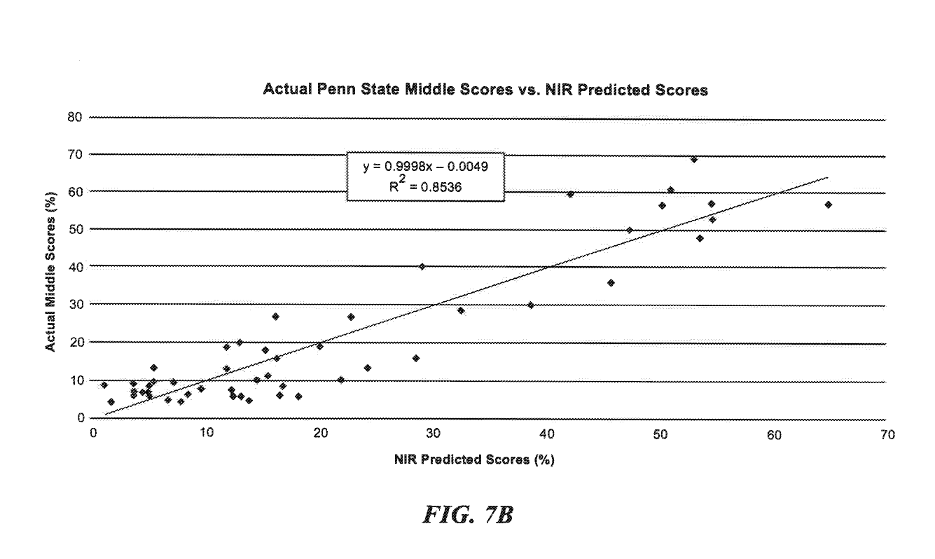

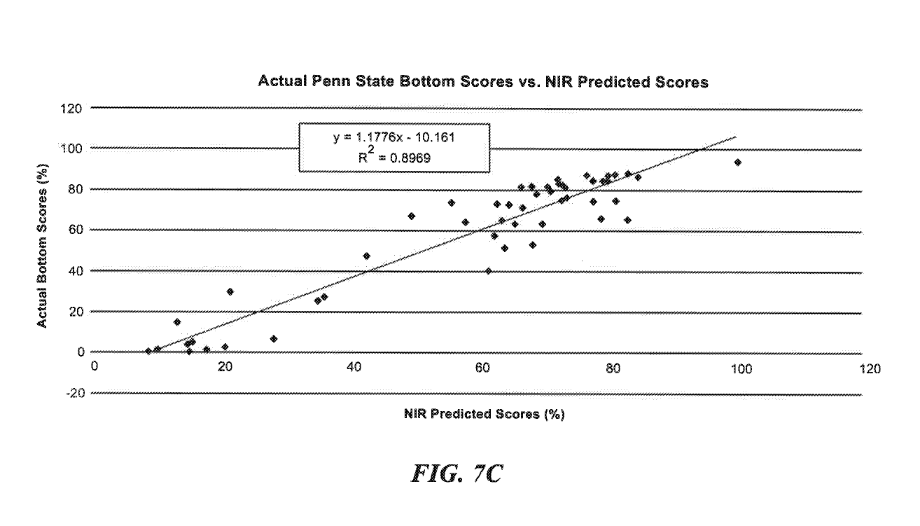

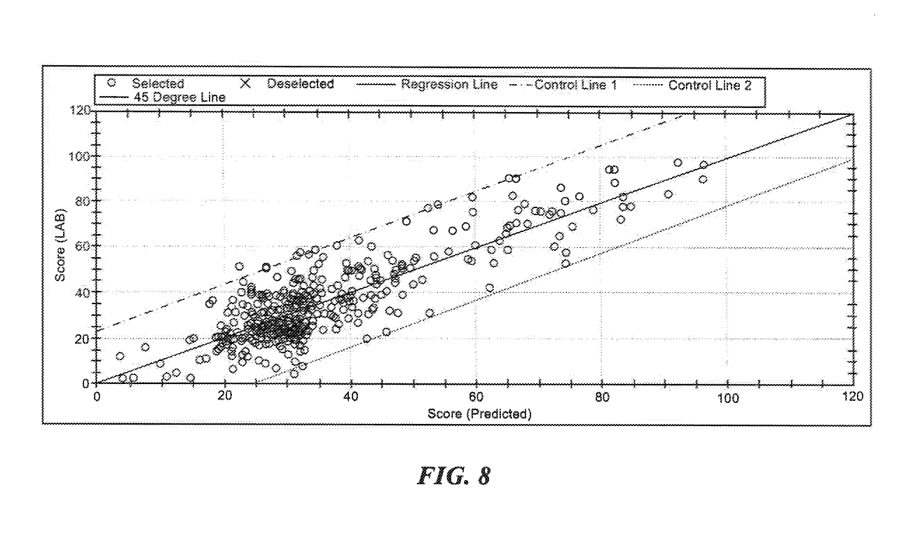

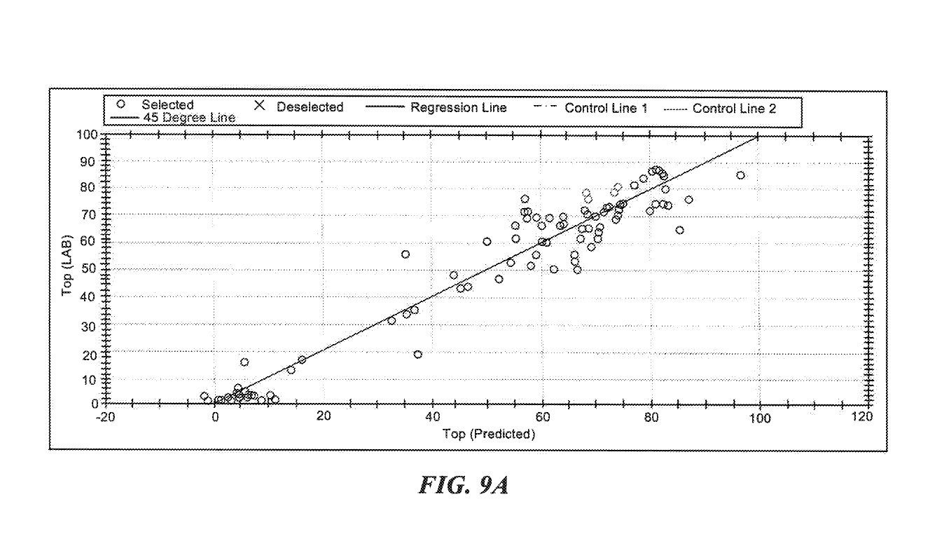

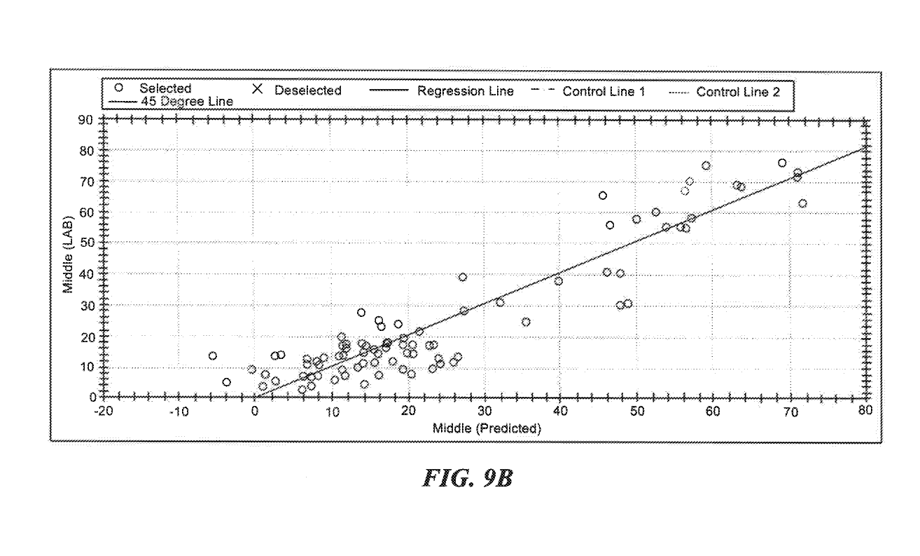

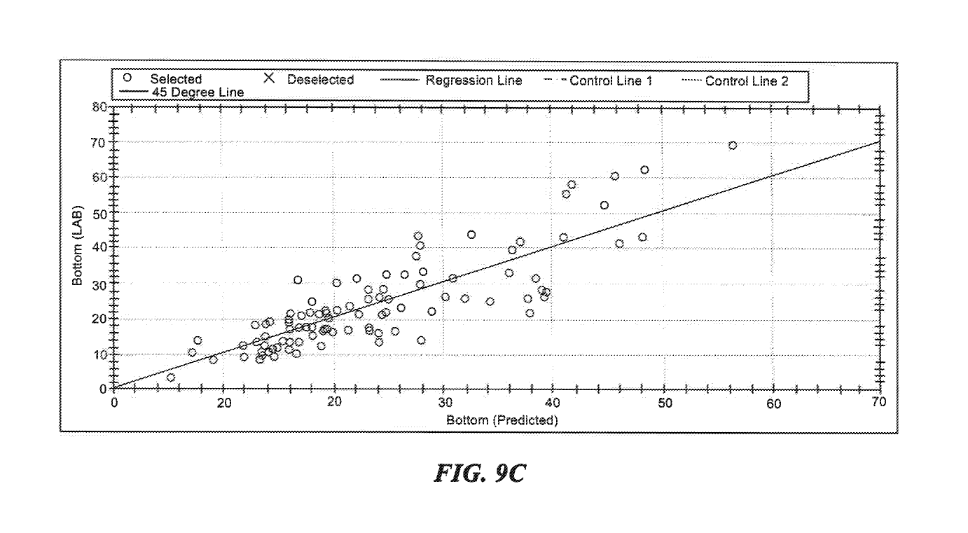

The graphs of FIG. 6 and FIGS. 7A through 7C illustrate the correlation between actual particles and NIR predicated scores for the testing sets for both Alternative Particle Score method (FIG. 6) and the Penn State Particle Separator method (FIGS. 7A through 7C).

As shown in FIG. 6, for the Alternative Particle Scorer method, the slope of the linear regression between actual and predicted results is 1.00. Also as shown in FIG. 6, for the Alternative Particle Scorer method, R2 as used to express the explained variance (in percentage) by the regression model is 0.67. As shown in the graph of FIG. 6, verification of the calibration is relatively good.

As shown in FIG. 7A, for the Penn State Particle Separator method top sieve, the slope of the linear regression between actual and predicted results is 1.00. Also as shown in FIG. 7A, for the Penn State Particle Separator method top sieve, R2 as used to express the explained variance (in percentage) by the regression model is 0.67. Also as shown in the graph of FIG. 7A, verification of the calibration is relatively good.

As shown in FIG. 7B, for the Penn State Particle Separator method middle sieve, the slope of the linear regression between actual and predicted results is 0.999. Also as shown in FIG. 7B, for the Penn State Particle Separator method middle sieve, R2 as used to express the explained variance (in percentage) by the regression model is 0.85. Also as shown in the graph of FIG. 7B, verification of the calibration is relatively good.

As shown in FIG. 7C, for the Penn State Particle Separator method bottom sieve, the slope of the linear regression between actual and predicted results is 1.18. Also as shown in FIG. 7C, for the Penn State Particle Separator method bottom sieve, R2 as used to express the explained variance (in percentage) by the regression model 0.879. Also as shown in the graph of FIG. 7C, verification of the calibration is relatively good.

The NIR calibration was then verified (i.e., adjusted for specific individual instrument bias).

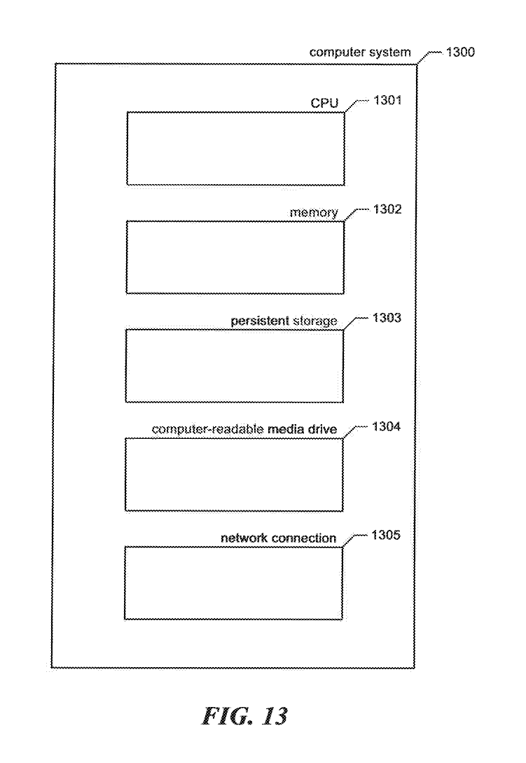

FIG. 13 is a flow diagram illustrating some of the components that may be incorporated in at least some of the computer systems and other devices on which the system operates and interacts with in some examples. In various examples, these computer systems and other devices 1300 can include server computer systems, desktop computer systems, laptop computer systems, netbooks, tablets, mobile phones, personal digital assistants, televisions, cameras, automobile computers, electronic media players, and/or the like. In various examples, the computer systems and devices include one or more of each of the following: a central processing unit (“CPU”) 1301 configured to execute computer programs; a computer memory 1302 configured to store programs and data while they are being used, including a multithreaded program being tested, a debugger, an operating system including a kernel, and device drivers; a persistent storage device 1303, such as a hard drive or flash drive configured to persistently store programs and data; a computer-readable storage media drive 1304, such as a floppy, flash, CD-ROM, or DVD drive, configured to read programs and data stored on a computer-readable storage medium, such as a floppy disk, flash memory device, a CD-ROM, a DVD; and a network connection 1305 configured to connect the computer system to other computer systems to send and/or receive data, such as via the Internet, a local area network, a wide area network, a point-to-point dial-up connection, a cell phone network, or another network and its networking hardware in various examples including routers, switches, and various types of transmitters, receivers, or computer-readable transmission media. While computer systems configured as described above may be used to support the operation of the disclosed techniques, those skilled in the art will readily appreciate that the disclosed techniques may be implemented using devices of various types and configurations, and having various components. Elements of the disclosed systems and methods may be described in the general context of computer-executable instructions, such as program modules, executed by one or more computers or other devices. Generally, program modules include routines, programs, objects, components, data structures, and/or the like configured to perform particular tasks or implement particular abstract data types and may be encrypted. Moreover, the functionality of the program modules may be combined or distributed as desired in various examples. Moreover, display pages may be implemented in any of various ways, such as in C++ or as web pages in XML (Extensible Markup Language), HTML (HyperText Markup Language), JavaScript, AJAX (Asynchronous JavaScript and XML) techniques or any other scripts or methods of creating displayable data, such as the Wireless Access Protocol (“WAP”).

The following discussion provides a brief, general description of a suitable computing environment in which the invention can be implemented. Although not required, aspects of the invention are described in the general context of computer-executable instructions, such as routines executed by a general-purpose data processing device, e.g., a server computer, wireless device or personal computer. Those skilled in the relevant art will appreciate that aspects of the invention can be practiced with other communications, data processing, or computer system configurations, including: Internet appliances, hand-held devices (including personal digital assistants (PDAs)), wearable computers, all manner of cellular or mobile phones (including Voice over IP (VoIP) phones), dumb terminals, media players, gaming devices, multi-processor systems, microprocessor-based or programmable consumer electronics, set-top boxes, network PCs, mini-computers, mainframe computers, and the like. Indeed, the terms “computer,” “server,” “host,” “host system,” and the like are generally used interchangeably herein, and refer to any of the above devices and systems, as well as any data processor.

Aspects of the invention can be embodied in a special purpose computer or data processor that is specifically programmed, configured, or constructed to perform one or more of the computer-executable instructions explained in detail herein. While aspects of the invention, such as certain functions, are described as being performed exclusively on a single device, the invention can also be practiced in distributed environments where functions or modules are shared among disparate processing devices, which are linked through a communications network, such as a Local Area Network (LAN), Wide Area Network (WAN), or the Internet. In a. distributed computing environment, program modules may be located in both local and remote memory storage devices.

Aspects of the invention may be stored or distributed on computer-readable storage media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the invention may be distributed over the Internet or over other networks (including wireless networks), on a propagated signal on a computer-readable propagation medium or a computer-readable transmission medium (e.g., an electromagnetic wave(s), a sound wave, etc.) over a period of time, or they may be provided on any analog or digital network (packet switched, circuit switched, or other scheme). Non-transitory computer-readable media include tangible media and storage media, such as hard drives. CD-ROMs, DVD-ROMS, and memories, such as ROM, RAM, and Compact Flash memories that can store instructions. Signals on a carrier wave such as an optical or electrical carrier wave are examples of transitory computer-readable media.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the invention is not intended to be exhaustive or to limit the invention to the precise form disclosed above. While specific examples for the invention are described above for illustrative purposes, various equivalent modifications are possible within the scope of the invention, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

The teachings of the invention provided herein can be applied to other systems, not necessarily the system described above. The elements and acts of the various examples described above can be combined to provide further implementations of the invention. Some alternative implementations of the invention may include not only additional elements to those implementations noted above, but also may include fewer elements.

Any patents and applications and other references noted above, including any that may be listed in accompanying filing papers, are incorporated herein by reference. Aspects of the invention can be modified, if necessary, to employ the systems, functions, and concepts of the various references described above to provide yet further implementations of the invention.

These and other changes can be made to the invention in light of the above Detailed Description. While the above description describes certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

To reduce the number of claims, certain aspects of the invention are presented below in certain claim forms, but the applicant contemplates the various aspects of the invention in any number of claim forms. For example, while only one aspect of the invention is recited as a means-plus-function claim under 35 U.S.C. §112(f), other aspects may likewise be embodied as a means-plus-function claim, or in other forms, such as being embodied in a computer-readable medium. (Any claims intended to be treated under 35 U.S.C. §112(f) will begin with the words “means for”, but use of the term “for” in any other context is not intended to invoke treatment under 35 U.S.C. §112(f).) Accordingly, the applicant reserves the right to pursue additional claims after filing this application to pursue such additional claim forms, in either this application or in a continuing application.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. When the claims use the word “or” in reference to a list of two or more items, that word covers all of the following interpretations of the word: any of the items in the list, all of the items in the list, and any combination of the items in the list.

The above detailed descriptions of embodiments of the invention are not intended to be exhaustive or to limit the invention to the precise form disclosed above. Although specific embodiments of, and examples for, the invention are described above for illustrative purposes, various equivalent modifications are possible within the scope of the invention, as those skilled in the relevant art will recognize. For example, while steps are presented in a given order, alternative embodiments may perform steps in a different order. The various embodiments described herein can also be combined to provide further embodiments.

In general, the terms used in the following claims should not be construed to limit the invention to the specific embodiments disclosed in the specification, unless the above detailed description explicitly defines such terms. While certain aspects of the invention are presented below in certain claim forms, the inventors contemplate the various aspects of the invention in any number of claim forms. Accordingly, the inventors reserve the right to add additional claims after filing the application to pursue such additional claim forms for other aspects of the invention.

1. A method for developing a calibration for a near infrared reflectance spectrophotometer to predict the particle score of an ingredient, the method comprising:
a. sorting a plurality of plant matter samples by size by passing the plurality of plant matter samples through a screen and subsequently calculating a particle score for the plurality of plant matter samples based on the number of plant matter samples passing through the screen;
b. measuring the absorbance or reflectance of the plurality of plant matter samples using the spectrophotometer; and
c. correlating the particle score from step (a) with the measured absorbance or reflectance from step (b).

2. The method of claim 1, wherein correlating the particle score further comprises constructing a curve by correlating the particle score from step (a) with the measured absorbance or reflectance from step (b).

3. The method of claim 2, wherein using the spectrophotometer further comprises at least one of using a using a near-infrared spectrophotometer, a near infrared reflectance spectrophotometer, a near infrared transmission spectrophotometer, an ultra violet spectrophotometer, a visible spectrophotometer, a Fourier transform near infrared spectrophotometer, a Raman spectrophotometer, and a mid-infrared spectrophotometer.

4. The method of claim 3, wherein sorting the plurality of plant matter samples by size further comprises measuring the chop length of each of the plurality of plant matter samples.

5. The method of claim 4, wherein correlating the particle score from step (a) with the measured absorbance or reflectance from step (b) further comprises conducting a regression analysis.

6. The method of claim 5, wherein conducting the regression analysis further comprises at least one of multiple linear regression (MLR), principal component regression (PCR), partial least squares (PLS), artificial neural networks (ANN), locally weighted regression (LWR), and support vector machines (SVM).

7. The method of claim 6, wherein passing the plurality of plant matter samples through the screen further comprises passing the samples through a particle separator having an upper sieve with a pore size of 0.75 inches or less, a middle sieve with a pore size of 0.31 inches or less, a lower sieve with a pore size of 0.16 inches or less, and a bottom pan.

8. The method of claim 7, wherein passing the plurality of plant matter samples through the screen further comprises passing the plurality of plant matter samples through a particle separator comprising a Penn State Particle Separator.

9. The method of claim 6, wherein passing the plurality of plant matter samples through a screen further comprises passing the plurality of plant matter samples through a particle separator comprising an Alternative Particle Scorer.

10. The method of claim 8, wherein calculating the particle score further comprises calculating the particle score according to Penn State Particle Separator method.

11. The method of claim 9, wherein passing the plurality of plant matter samples through the Alternative Particle Scorer further comprises passing the plurality of plant matter samples through a screen with a size of 0.065 inches or less.

12. The method of claim 11, wherein calculating the particle score further comprises calculating the particle score according to Alternative Particle Scorer method.

13. An NIR calibration for predicting particle score for a dry ingredient, the calibration produced by a method comprising:
a. sorting a plurality of forage samples by chop length by passing the plurality of forage samples through a particle separator having at least one screen and subsequently calculating a particle score for the plurality of forage samples based on the weight of the plurality of forage samples passing through the screen;
b. measuring the absorbance or reflectance of the plurality of forage samples using the spectrophotometer; and
c. correlating the particle score from step (a) with the measured absorbance or reflectance from step (b).

14. The NIR calibration of claim 13, wherein correlating the particle score from step (a) with the measured absorbance or reflectance from step (b) further comprises conducting a regression analysis comprising at least one of multiple linear regression (MLR), principal component regression (PCR), partial least squares (PLS), artificial neural networks (ANN), locally weighted regression (LWR), and support vector machines (SVM).

15. The NIR calibration of claim 14, wherein passing the plurality of forage samples through the screen further comprises passing the plurality of forage samples through a particle separator comprising at least one of a Penn State Particle Separator and an Alternative Particle Scorer.

16. The NIR calibration of claim 15, wherein calculating the particle score further comprises calculating the particle score according to at least one of Penn State Particle Separator method and Alternative Particle Scorer method.

17. A method for formulating a feed, the method comprising:
a. calibrating a near infrared reflectance spectrophotometer, comprising:
i. sorting a plurality of forage samples by chop length by passing the plurality of forage samples through a particle separator having a screen and subsequently calculating a particle score for plurality of forage samples based on the number of samples passing through the screen;
ii. measuring the absorbance or reflectance of the plurality of forage samples using the spectrophotometer;
iii. correlating the particle score from step (i) with the measured absorbance or reflectance from step (ii);
b. predicting the particle score of a total mixed ration using a near infrared reflectance spectrophotometer correlated according to step (iii);
c. formulating a feed based on the particle score of the total mixed ration.

18. The method of claim 17, further comprising mixing ingredients with the total mixed ration.

19. The method of claim 18, further feeding the ingredients and the total mixed ration to an animal.

20-27. (canceled)
